(19) United States
(12) Patent Application Publication
(10) Pub. No.: US 2026/0244262 A1
(43) Pub. Date: Aug. 20, 2026

(54) LONGITUDINAL HUMAN SENSORY MONITORING SYSTEM WITH PERSISTENT USER RECORDS, STATE CONSTRUCTION, CHANGE DETECTION, AND ACTION DETERMINATION

(71) Applicant: Datar Sahi, Marina del Rey, CA (US)

(72) Inventor: Datar Sahi, Marina del Rey, CA (US)

(21) Appl. No.: 19/641,791

(22) Filed: Apr. 8, 2026

Related U.S. Application Data
(63) Continuation-in-part of application No. 18/957,791, filed on Nov. 24, 2024.

(51) Int. Cl. G06F 3/01; G16H 40/67

(52) U.S. Cl. CPC G06F 3/011; G16H 40/67

(57) ABSTRACT

A computer-implemented system for longitudinal human sensory monitoring is disclosed. A mobile device subsystem guides testing and obtains sensory results for one or more of smell, taste, hearing, vision, and touch at multiple time points. A remote computing subsystem stores each result as an atomic, immutable test event in a persistent user record, constructs sensory snapshots as time-bounded user states from selected events, and designates baseline and updated states. The subsystem compares a current sensory snapshot with at least one prior snapshot or baseline to detect change in sensory status over time, including direction, magnitude, and rate of change, and classifies change into categories such as improvement, stability, or decline. Based on detected change and optional user-reported or external physiological data, the system determines one or more actions, including recommendations, interventions, scheduling or alert outputs, and iteratively updates actions within a continuous monitoring loop

### CROSS-REFERENCE TO RELATED APPLICATION

This application is a continuation-in-part (CIP) of a prior application – 18957791, filed 11-24-2025 - directed to a system for comprehensive, application-guided, human sensory profiling. The disclosure of the parent application is incorporated herein by reference to the extent permitted and to the extent not inconsistent with the present disclosure.

### TECHNICAL FIELD

The present invention relates generally to systems and methods for measurement of human sensory performance and, more particularly, to application-guided sensory testing systems that obtain sensory data for one or more human senses, maintain persistent user-specific sensory records over time, construct sensory states from time-stamped sensory events, detect longitudinal change in sensory function, and determine actions based on detected change.

### BACKGROUND

Sensory testing systems can assess one or more of vision, hearing, smell, taste, and touch. Existing systems may generate scores or comparative rankings for a user based on a testing session and, in some cases, may compare the user's results to results from a broader population.

However, many sensory assessment systems remain focused on isolated testing sessions, static scoring, or comparisons to external populations. Such systems may not preserve sensory measurements as immutable user-specific events across time, may not construct defined sensory states from temporally related events, and may not provide a rule-based framework for detecting longitudinal change and determining user-specific actions in response to such change.

A need therefore exists for a sensory monitoring system that supports persistent individual tracking over multiple time points, preserves underlying sensory measurements for auditability and re-analysis, distinguishes between atomic event data and constructed state representations, supports incomplete and complete data conditions, detects change relative to baseline or prior states, and determines one or more actions based on detected change.

### BRIEF DESCRIPTION OF INVENTION

In one aspect, a computer-implemented sensory monitoring system includes a mobile device subsystem and a remote computing subsystem communicating over a network. The mobile device subsystem obtains sensory test results for one or more of smell, taste, hearing, vision, and touch at different times. The remote computing subsystem stores each sensory test result as an atomic, immutable test event in a persistent user record.

In some embodiments, the remote computing subsystem constructs a sensory snapshot from a selected set of atomic, immutable test events. The sensory snapshot represents a time-bounded sensory state of the user and may correspond to a multi-sense assessment, a partial assessment, or another defined temporal grouping of sensory information.

In some embodiments, the remote computing subsystem compares a current sensory snapshot to at least one prior sensory snapshot or to a baseline state for the user to determine change in sensory status over time. The system may determine a direction, magnitude, rate, or pattern of change and may classify the change as improvement, stability, decline, or another status category.

In some embodiments, the system determines at least one action based on detected change. The action may include a recommendation, suggested next step, intervention, protocol, alert, scheduling action, or communication output. The mapping from detected change to action may be rule-based, model-based, or hybrid.

In some embodiments, the system stores partial sensory data before all required inputs for a defined snapshot are present, supports limited outputs from partial data, and defers generation of a composite output until predefined criteria are met.

In some embodiments, the system incorporates secondary data, such as user-reported symptoms, perceived changes, adherence information, laboratory data, imaging data, or clinical assessments, to influence interpretation of sensory change or action determination.

In some embodiments, the system operates as a repeated loop in which sensory data is collected, user state is constructed or updated, change is detected relative to prior data, and one or more actions are determined and then updated as new data becomes available.

In some embodiments, the system also generates cohort-based or population-based comparisons, but individual longitudinal tracking remains a primary basis for user-specific interpretation and action determination.

### BRIEF DESCRIPTION OF THE DRAWINGS

FIG. 1 is a block diagram showing an example of a mobile device subsystem that guides a user through sensory testing.

FIG. 2 is a block diagram showing an example of a remote computing subsystem that stores sensory data, constructs sensory states, performs longitudinal comparisons, and determines actions.

FIG. 3 is a diagram showing an example persistent user record comprising atomic sensory test events, baseline data, snapshots, and derived state information over time.

FIG. 4 is a diagram showing an example relationship between partial sensory inputs, completion criteria, snapshot generation, and deferred composite output generation.

FIG. 5 is a flow diagram showing an example rule-based action determination process responsive to detected longitudinal sensory change.

### DETAILED DESCRIPTION OF INVENTION

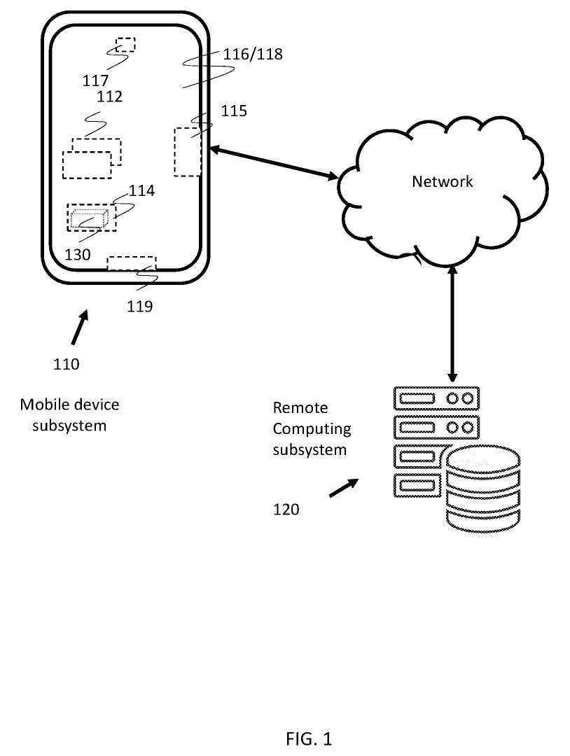

In one embodiment, the system includes a user-facing subsystem, such as a mobile device subsystem 110, and a remote computing subsystem 120, as schematically illustrated in FIG. 1. The mobile device subsystem 110 can present testing content, receive responses, guide use of external sensory test kits or accessories, store temporary data, display sensory outputs, and communicate with the remote computing subsystem 120 through one or more wired or wireless networks.

As shown in FIG. 1, the mobile device subsystem 110 may include one or more processors 112, memory 114, a display 116, a touchscreen or other user input interface 118, an audio output device 119, an optional camera or sensor interface 117, and a communication interface 115. Program instructions executed by the mobile device subsystem 110 can guide a user through sensory testing procedures for vision, hearing, smell, taste, and touch.

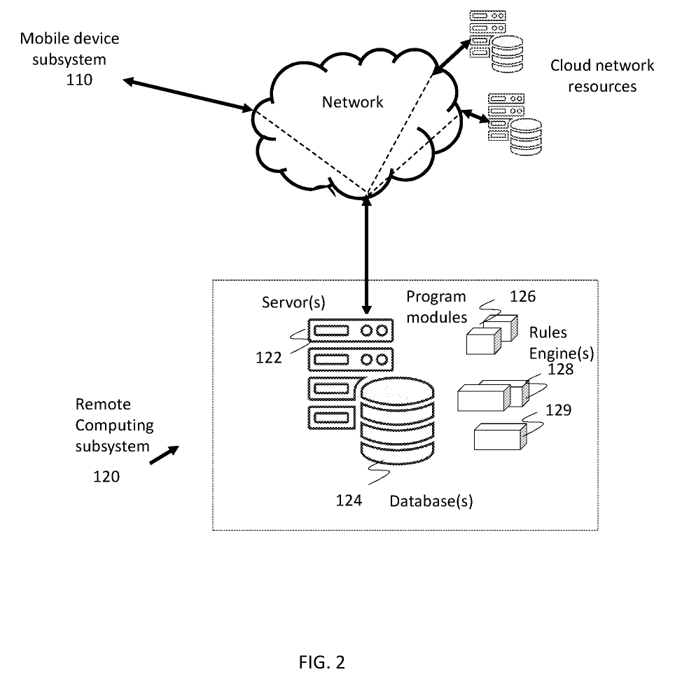

As schematically illustrated in FIG. 2, the remote computing subsystem 120 may include one or more servers 122, cloud computing resources, a database 124, program modules 126, rules engines including a rules engine 128, analytics engines and machine-learning modules 129, and interfaces 125 for receiving sensory data and delivering outputs. In some embodiments, the remote computing subsystem 120 performs event storage, state construction, longitudinal comparison, change classification, action determination, reporting, and optional cohort comparison.

Sensory data may be obtained from any suitable sensory testing modality. For example, visual function may be tested through on-screen image, color, contrast, or pattern discrimination exercises. Hearing function may be tested through reproduced sounds, tones, speech, or audio-visual association tasks. Smell, taste, and touch may be tested through application-guided use of physical kits, cards, strips, textures, or other test articles. In some implementations, as further represented in FIG. 1, the mobile device subsystem 110 executes one or more sensory testing modules 130 that present testing content and receive user responses for one or more of the sensory modalities.

Each completed sensory test or sensory measurement is stored as an atomic test event. In preferred embodiments, an atomic test event is immutable after creation. The event may include, for example, a user identifier, timestamp, sense type, test type, raw result data, processed score data, device data, session data, quality indicators, and metadata sufficient to support later interpretation.

Because the atomic test event is preserved without modification, later retesting does not overwrite earlier information. Instead, a later retest creates a new atomic event, thereby preserving a longitudinal record of the user's sensory measurements. This event-level immutability supports traceability, auditability, and alternate re-analysis approaches.

For each user, the remote computing subsystem maintains a persistent user record. The persistent user record may include user identifiers, demographic data, baseline designations, time-stamped sensory test events, snapshot identifiers, derived state data, change metrics, action history, adherence information, user-reported information, external physiological data, and communication history.

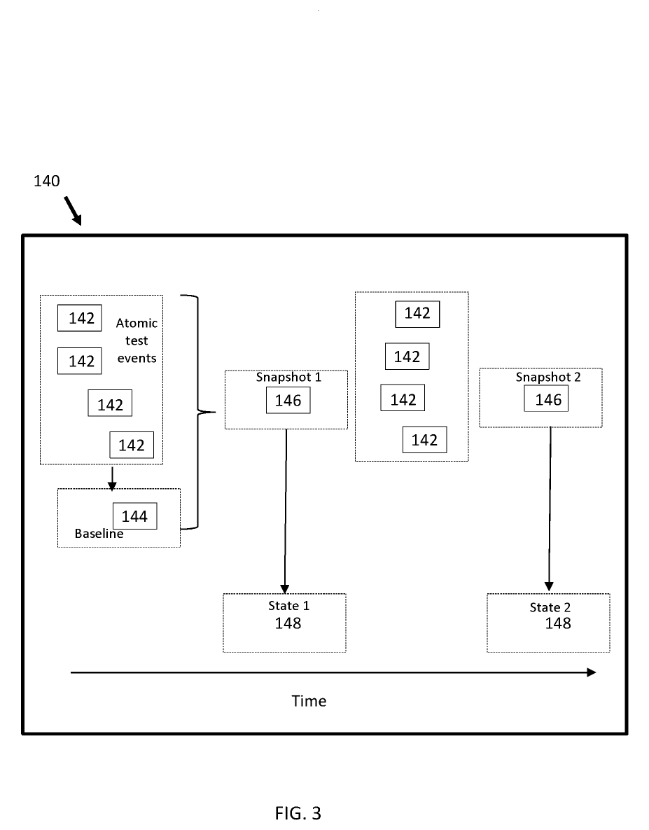

The persistent user record may designate one or more baseline states. In some embodiments, an initial sensory assessment is designated as a baseline. In other embodiments, a baseline may be selected according to one or more rules, such as the first sufficiently complete multi-sense assessment, the earliest qualifying snapshot within a date range, or a baseline reset after an intervention or clinical milestone. An example persistent user record 140 including atomic sensory test events 142, baseline data 144, sensory snapshots 146, and derived state information 148 over time is shown in FIG. 3.

In some embodiments, the remote computing subsystem constructs a sensory snapshot representing a defined time-bounded state of the user. A sensory snapshot may reference one or more atomic events that fall within a selected time window, session boundary, assessment episode, or other defined grouping criterion.

A sensory snapshot may include events for all five senses, for fewer than five senses, or for any predefined set of senses required by a given workflow. The system may define one or more completion criteria for a snapshot, such as presence of required senses, minimum data quality, a maximum time gap between included events, or a combination of such criteria.

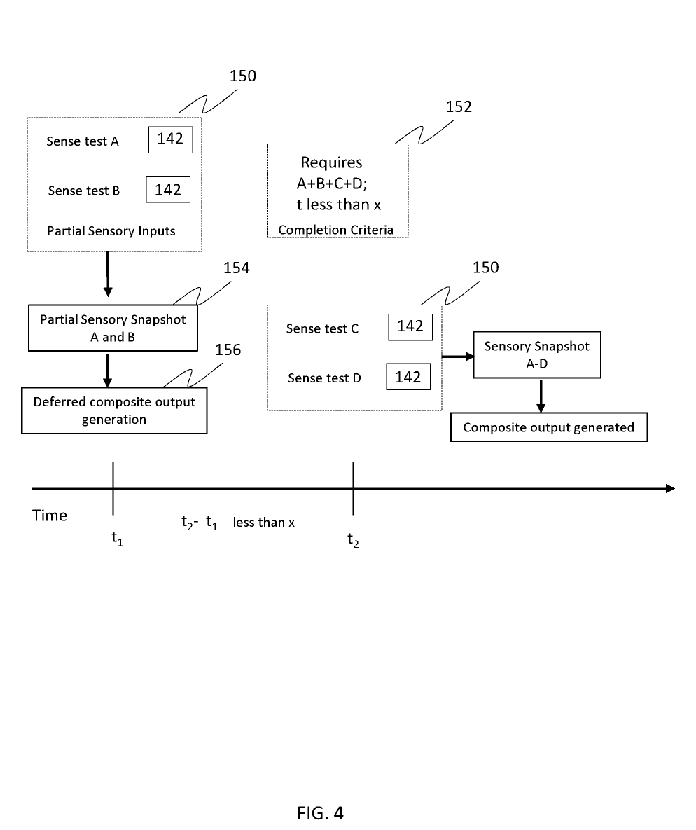

If snapshot completion criteria are not met, the underlying data may still be stored in the persistent user record. The system may defer generating the snapshot, may generate a partial snapshot, or may produce one or more limited outputs that are clearly distinguished from a full composite output. FIG. 4 depicts an example relationship between partial sensory inputs 150, completion criteria 152, snapshot generation 154, and deferred composite output generation 156.

In some embodiments, once a snapshot is created it is not modified. Instead, later data may lead to creation of a new snapshot or another derivative structure. In other embodiments, the system may permit constrained re-computation under defined conditions while preserving prior versions or audit records.

The system may support incomplete data states. For example, a user may complete hearing and smell testing during a first session and complete vision and taste testing during a later session. The remote computing subsystem may store the available atomic events, track which required inputs are still absent, and determine whether a partial output may appropriately be generated. As further illustrated in FIG. 4, partial sensory inputs 150 may be retained pending satisfaction of the completion criteria 152 for generation of a composite output.

A limited output generated from partial data may include a per-sense result, a provisional state indicator, a reminder, a completion prompt, or a restricted recommendation. In some embodiments, the system suppresses generation of a composite score, composite classification, or full recommendation package until required completion criteria are satisfied.

Once at least two relevant states are available, the remote computing subsystem may perform longitudinal comparison. A current state may be compared to an immediately prior state, to a baseline state, to a rolling historical window, or to multiple prior states.

Change detection may involve any suitable metric or logic. For example, the system may compute absolute differences, normalized differences, weighted multi-sense deltas, trends over multiple time points, rates of change, threshold crossings, or pattern detections across senses. Detected change may be classified into categories such as improvement, stability, decline, transient fluctuation, asymmetric change, or mixed-pattern change.

In some embodiments, change classification considers both magnitude and direction. In some embodiments, time spacing between assessments is also considered so that identical numerical changes over different intervals may be interpreted differently.

Based on detected change, the system may determine one or more actions. An action may include, without limitation, presenting a recommendation, scheduling a retest, changing a testing cadence, prompting a user to complete missing sensory inputs, recommending environmental or behavioral adjustments, suggesting a clinical consultation, selecting an intervention protocol, or transmitting an alert to another system or authorized recipient.

Action determination may be performed using one or more rules stored in a rules engine. By way of example, a threshold decline in one sense may trigger a retest recommendation, a repeated decline across multiple senses may trigger an elevated alert or protocol, and stable measurements over a defined interval may trigger a reduced monitoring frequency. In some embodiments, action determination additionally uses statistical models or machine-learning models, including optional model outputs, while retaining explicit rule constraints.

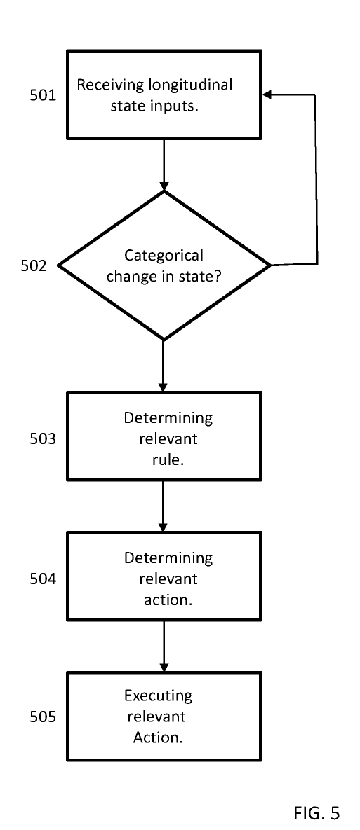

An example rule-based action determination process responsive to longitudinal state inputs (501) and detected longitudinal sensory change (502) is shown in FIG. 5, wherein when change is detected, a rules engine determines relevant rule (503) and relevant action (504), and the system executes the relevant action (505).

After an action is determined, the system may receive feedback. Feedback may include user-reported outcomes, user adherence to a recommendation or protocol, clinician input, further sensory measurements, or other follow-up information. The remote computing subsystem may use such feedback to update a current action, select a new action, modify monitoring cadence, or refine future decision-making.

In some embodiments, action determination is therefore iterative. As new sensory data or follow-up information becomes available, the system may re-evaluate the user's state and issue updated outputs. This repeated loop may continue throughout a monitoring period or indefinitely.

In some embodiments, the system receives secondary data in addition to sensory test data. Secondary data may include user-reported symptoms, perceived changes, medication adherence, sleep information, environmental exposure information, laboratory measurements, imaging results, physiological sensor data, and clinician assessments.

Secondary data may be used to contextualize sensory change, modify confidence in an interpretation, prioritize an action, suppress an action, or trigger an additional inquiry. In some embodiments, the system remains operable using sensory data alone, and the secondary data functions as optional augmentation rather than a required dependency.

The system may enforce one or more temporal rules. Examples include a maximum permitted interval between sensory events for a common snapshot, minimum spacing between repeated tests for a given sense, freshness thresholds for prior data used in comparison, and time-based decay or weighting rules for older measurements.

The system may also impose system constraints regarding modification or re-computation. For example, snapshots may be locked after creation, baseline designations may be preserved unless an authorized reset event occurs, and re-computation of prior outputs may be prevented or limited except under specified conditions.

In some embodiments, the system additionally compares a user's sensory data or state to a cohort or population distribution, including demographic cohorts defined by age, gender, geography, health condition, or other attributes. Such cohort analysis may provide percentile information, normative ranges, or subgroup-relative status.

However, in preferred embodiments of the present continuation-in-part disclosure, individual longitudinal tracking remains primary. Population or cohort comparison is used as a secondary contextual layer rather than as the sole basis for user-specific interpretation or action determination.

The system may operate as a continuous or repeated monitoring loop in which sensory data is collected, atomic events are stored, a state is constructed, change is detected relative to prior information, an action is determined, and later data is used to update state and action. The loop can be implemented at regular intervals, in response to user-initiated testing, in response to reminders, or in response to external triggers.

This loop-based architecture allows the system to move beyond static sensory scoring and toward ongoing longitudinal sensory monitoring with individualized outputs over time. After an action is determined, later sensory data or follow-up information may cause the system to re-enter the loop and update state and action.

By way of non-limiting example, a user may complete smell and hearing tests on a first day, vision and touch tests on a second day, and taste testing later that week. The remote computing subsystem stores each result as a separate atomic event. If a full multi-sense snapshot requires all five senses within a predefined window, the system may initially store the earlier events without generating a full composite output, for example in the deferred-output arrangement (156) illustrated in FIG. 4.

When the required set of events is present within the applicable window, the system constructs a baseline snapshot. At a later date, the user completes another set of tests and the system constructs a new snapshot, compares the new snapshot to the baseline snapshot, detects a decline in smell and stability in hearing and vision, classifies the overall longitudinal change, and determines an action such as a retest recommendation, a prompt for additional information, or another protocol.

If the user later reports improvement or completes additional testing, the system may incorporate that feedback and update the determined action accordingly.

The system can be implemented using smartphones, tablets, laptops, kiosks, dedicated testing devices, or distributed combinations of such devices. Remote computing functionality may be implemented by one or more cloud servers, local servers, edge devices, or hybrid architectures.

The specific sensory tests, state-construction rules, comparison logic, classification rules, and action mappings may vary according to clinical, wellness, research, or consumer applications. One or more senses may be emphasized, omitted, or supplemented in different embodiments.

Although certain embodiments herein describe snapshots and states as discrete structures, analogous implementations may use graphs, linked records, versioned objects, timelines, or other data organizations while preserving the distinction between underlying sensory events and derived state representations.

Accordingly, the disclosed embodiments are illustrative and not limiting. Variations, combinations, and modifications may be made without departing from the scope of the invention as defined by the claims.

What is claimed is:

1. A computer-implemented sensory monitoring system for a user, comprising:
a mobile device subsystem comprising at least one processor, memory, a display, a user input interface, and a network interface;
a remote computing subsystem in communication with the mobile device subsystem over a network;
sensory testing logic executable by the mobile device subsystem to obtain sensory test results for at least one of smell, taste, hearing, vision, or touch at a plurality of different times; and
record-management and analysis logic executable by the remote computing subsystem and configured to:
store each sensory test result as a respective atomic, immutable test event associated with the user, a timestamp, a sense type, and result data;
maintain, for the user, a persistent user record comprising a plurality of the atomic, immutable test events over time;
construct from a selected set of the atomic, immutable test events a sensory snapshot representing a time-bounded sensory state of the user;
compare a current sensory snapshot with at least one prior sensory snapshot or with a baseline state for the user to determine a change in sensory status over time; and
determine at least one action for the user based on the determined change in sensory status.

2. The system of claim 1, wherein the remote computing subsystem designates an initial sensory state as the baseline state and designates one or more later sensory states as updated states for the user.

3. The system of claim 1, wherein each atomic, immutable test event is preserved without modification after it has been created, and a retest for a same sense produces a new atomic, immutable test event rather than overwriting an earlier test event.

4. The system of claim 1, wherein the sensory snapshot references a plurality of atomic, immutable test events corresponding to different senses collected within a defined time window.

5. The system of claim 4, wherein the remote computing subsystem defers generation of the sensory snapshot until a required set of sensory inputs is present within the defined time window.

6. The system of claim 1, wherein the remote computing subsystem stores partial sensory inputs in the persistent user record and generates a limited output based on the partial sensory inputs while withholding generation of a composite output until predefined completion criteria are satisfied.

7. The system of claim 1, wherein determining the change in sensory status over time comprises determining at least one of a direction of change, a magnitude of change, or a rate of change between time points.

8. The system of claim 7, wherein the remote computing subsystem classifies the change in sensory status as improvement, stability, or decline.

9. The system of claim 1, wherein the at least one action comprises a recommendation, a suggested next step, an intervention, or a protocol selected according to a rule-based mapping between detected change and determined action.

10. The system of claim 1, wherein the record-management and analysis logic operates in a continuous update loop in which newly received sensory test data updates the persistent user record, produces an updated sensory state, and causes re-determination of the at least one action.

11. The system of claim 1, wherein the remote computing subsystem further receives user-reported data comprising symptoms, perceived changes, or adherence information, and uses the user-reported data in determining the at least one action.

12. The system of claim 1, wherein the remote computing subsystem further receives external physiological data comprising at least one of laboratory data, imaging data, or clinical assessment data, and uses the external physiological data to influence interpretation of the change in sensory status or determination of the at least one action.

13. The system of claim 1, wherein the remote computing subsystem updates the at least one action based on feedback comprising user-reported outcomes, adherence to a prior action, or follow-up sensory measurements.

14. The system of claim 1, wherein the remote computing subsystem enforces at least one temporal rule comprising a time window for data aggregation, an interval between assessments, or a timing constraint affecting interpretation of sensory data.

15. The system of claim 1, wherein the remote computing subsystem restricts re-computation of a previously generated sensory snapshot under defined conditions.

16. The system of claim 1, wherein the remote computing subsystem is further configured to compare the user with a demographic cohort or a population distribution, wherein such comparison is secondary to said comparing of the current sensory snapshot with the at least one prior sensory snapshot or the baseline state for the user.

17. A computer-implemented method for longitudinal sensory monitoring of a user, comprising:
receiving, from a mobile device subsystem, sensory test results for at least one of smell, taste, hearing, vision, or touch at multiple time points;
storing each sensory test result as a respective atomic, immutable test event in a persistent user record for the user;
constructing, from a selected set of the atomic, immutable test events, a sensory snapshot representing a time-bounded sensory state of the user;
comparing a current sensory snapshot with a prior sensory snapshot or a baseline state to detect a change in sensory status over time; and
determining, by a computing system, at least one action for the user based on the detected change in sensory status.

18. The method of claim 17, further comprising storing partial sensory data before all required sensory inputs for the sensory snapshot are available, and deferring generation of a composite output until a predefined completion condition is satisfied.

19. The method of claim 17, wherein detecting the change in sensory status comprises classifying the change as improvement, stability, or decline based on direction and magnitude of change between time points.

20. The method of claim 17, further comprising repeating a loop in which new sensory test data is collected, the persistent user record is updated, a new sensory state is constructed, the change in sensory status is re-evaluated, and the at least one action is updated.

21. The method of claim 17, further comprising incorporating user-reported data or external physiological data into interpretation of the detected change in sensory status or into determination of the at least one action.

22. A non-transitory computer-readable medium storing instructions that, when executed by one or more processors, cause a computing system to:
maintain, for each of a plurality of users, a persistent user record comprising atomic, immutable sensory test events stored across multiple time points;
construct for a selected user a sensory snapshot from a selected set of the atomic, immutable sensory test events, the sensory snapshot representing a time-bounded sensory state;
compare a current sensory snapshot with at least one prior sensory snapshot or a baseline state for the selected user to detect change over time; and
determine an action for the selected user based on the detected change over time.

23. The non-transitory computer-readable medium of claim 22, wherein the instructions further cause the computing system to maintain previous sensory snapshots without modification after creation.

24. The non-transitory computer-readable medium of claim 22, wherein the instructions further cause the computing system to generate a cohort-based comparison for the selected user in addition to the detected change over time, while preserving individual longitudinal tracking as a primary analysis mode.

25. The system of claim 1, wherein the system measures sensory function across a plurality of senses, tracks changes over time at an individual-user level, constructs user states according to predefined rules, detects change across time, and determines actions based on the detected change.
